(19) United States
(12) Patent Application Publication
(10) Pub. No.: US 2013/0310629 A1
(43) Pub. Date: Nov. 21, 2013

(54) Methods, Systems, and Devices Relating to Wireless Power Transfer

(71) Applicant: SUNSHINE HEART COMPANY PTY LTD, (US)

(72) Inventor: Daniel M. Lafontaine, Plymouth, MN (US)

(73) Assignee: SUNSHINE HEART COMPANY PTY LTD, New South Wales (AU)

(21) Appl. No.: 13/799,597

(22) Filed: Mar. 13, 2013

Related U.S. Application Data
(60) Provisional application No. 61/610,173, filed on Mar. 13, 2012.

(51) Int. Cl. A61M 1/12

(52) U.S. Cl. CPC A61M 1/127
USPC 600/16

(57) ABSTRACT

The various embodiments disclosed herein relate to transcutaneous energy transfer systems comprising an internal coil positioned within a cavity of a patient and an external coil inductively coupled to the internal coil. The systems can be coupled to any implantable medical devices, such as, for example, a heart assist device.

### CROSS-REFERENCE TO RELATED APPLICATION(S)

This application claims the benefit under 35 U.S.C. §119(e) to U.S. Provisional Patent Application No. 61/610,173, filed on Mar. 13, 2012, which is hereby incorporated herein by reference in its entirety.

### FIELD OF THE INVENTION

The various embodiments disclosed herein relate to methods and devices for transferring electrical power transcutaneously into a cavity of a patient to power electrical therapy devices, including, for example, heart assist devices in the patient's thoracic lung cavity.

### BACKGROUND OF THE INVENTION

Fully implanted electrical therapy devices have evolved from the battery powered pacemakers to new therapies that require higher levels of energy to be delivered to the body, including nerve stimulation, drug delivery, muscle stimulation (TENS), heart assist technologies, and heart replacement with an artificial heart. The evolution of battery technology has made it possible to implant low power medical devices for up to ten years of operation. However, most fully implanted high current devices are presently powered with percutaneous cables, because a safe high-power battery technology still does not exist. The cables deliver safe power to the implant, but can cause the patient significant discomfort and require maintenance to prevent infection, which occurs in approximately 40% of these implants.

The known use of transcutaneous energy transfer (TET) to power implanted medical devices can eliminate the cables and reduce the risk of infection. However, these prior art TET systems have not eliminated the risk of infection because the known systems are bulky and require a significant amount of surgery and implanted hardware. Also, the prior art technology requires close mechanical coupling for efficient energy transfer, which increases the power density and electromagnetic field exposure to the patient. This is undesirable, because high electromagnetic field exposure can cause the specific absorption rate to be exceeded for biologic tissue limits, and high power density can lead to localized heating of patient tissue, which can cause tissue necrosis.

There is a need in the art for an improved TET system.

### BRIEF SUMMARY OF THE INVENTION

Discussed herein are various embodiments relating to TET systems.

In Example 1, a transcutaneous energy transfer system comprises an internal coil sized to be positioned within a pleural cavity of a patient and an external coil configured to be positioned in proximity to the patient such that the external coil and the internal coil are inductively coupled. The internal coil is configured to be positioned in proximity with at least one lung within the pleural cavity.

Example 2 relates to the system according to Example 1, wherein the internal coil has a diameter of at least about 6 cm.

Example 3 relates to the system according to Example 1, wherein the internal coil is positioned substantially against an inner wall of the pleural cavity.

Example 4 relates to the system according to Example 1, further comprising a self-expanding structure operably coupled to the internal coil.

Example 5 relates to the system according to Example 4, wherein the self-expanding structure is made of a shape memory material.

Example 6 relates to the system according to Example 4, wherein the self-expanding structure is configured to expand such that the internal coil is in contact with an inner wall of the pleural cavity.

Example 7 relates to the system according to Example 4, wherein the self-expanding structure comprises at least one insulated connector configured to prevent formation of a competing electrical circuit.

Example 8 relates to the system according to Example 1, wherein the internal coil is configured to be positioned around the at least one lung.

Example 9 relates to the system according to Example 1, wherein the external coil comprises a shoulder strap cushion.

Example 10 relates to the system according to Example 1, wherein the external coil is integrated into a shoulder strap, backpack, bag, vest, shirt, jacket, bed, chair, or car seat.

In Example 11, a transcutaneous energy transfer system operably coupled to a heart assist system comprises an internal coil sized to be positioned within a pleural cavity of a patient, a compliance chamber associated with the internal coil, and an external coil configured to be positioned in proximity to the patient such that the external coil and the internal coil are inductively coupled.

Example 12 relates to the system according to Example 11, wherein the compliance chamber is coupled to the internal coil.

Example 13 relates to the system according to Example 11, wherein the internal coil is positioned within the compliance chamber.

Example 14 relates to the system according to Example 11, wherein the compliance chamber is an expandable compliance chamber having an inflated configuration and a deflated configuration.

In Example 15, a transcutaneous energy transfer system comprises an internal coil sized to be positioned within a pleural cavity of a patient, a repeater coil, and an external coil. The repeater coil is configured to be positioned in proximity to the patient such that the repeater coil and the internal coil are inductively coupled. The external coil is configured to be positioned such that the external coil and the repeater coil are inductively coupled, whereby the external coil is inductively coupled to the internal coil.

Example 16 relates to the system according to Example 15, wherein the repeater coil is positioned in proximity to skin of the patient.

Example 17 relates to the system according to Example 15, wherein the internal coil has a diameter of at least about 6 cm.

Example 18 relates to the system according to Example 15, further comprising a self-expanding structure operably coupled to the internal coil.

Example 19 relates to the system according to Example 15, wherein the external coil comprises a shoulder strap cushion.

Example 20 relates to the system according to Example 15, wherein the external coil is integrated into a shoulder strap, backpack, bag, vest, shirt, jacket, bed, chair, or car seat. While multiple embodiments are disclosed, still other embodiments of the present invention will become apparent to those skilled in the art from the following detailed description, which shows and describes illustrative embodiments of the invention. As will be realized, the invention is capable of modifications in various obvious aspects, all without departing from the spirit and scope of the present invention. Accordingly, the drawings and detailed description are to be regarded as illustrative in nature and not restrictive.

### BRIEF DESCRIPTION OF THE DRAWINGS

FIG. 1 is a schematic depiction of a TET system, according to one embodiment.

FIG. 2 is a schematic depiction of an internal coil of another TET system, according to another embodiment.

FIG. 3A is a schematic depiction of an internal coil having a support structure, according to one embodiment.

FIG. 3B is a cross-section view of the internal coil of FIG. 3A, according to one embodiment.

FIG. 4 is a schematic depiction of a further TET system, according to a further embodiment.

FIG. 5A is a front view of an external coil of the TET system of FIG. 4, according to one embodiment.

FIG. 5B is a front view of an internal coil of the TET system of FIG. 4, according to one embodiment.

FIG. 6 is a schematic depiction of a internal coil of another TET system, according to one embodiment.

FIG. 7A is a cross-section view of an internal coil, according to another embodiment.

FIG. 7B is a cross-section view of the internal coil of FIG. 7A, according to another embodiment.

FIG. 7C is a cross-section view of the internal coil of FIG. 7A, according to another embodiment.

FIG. 8 is a is a schematic depiction of a TET system having an internal coil and a repeater coil, according to one embodiment.

FIG. 9 is a schematic depiction of a circuit description relating to a repeater coil, according to one embodiment.

FIG. 10 is a schematic depiction of the standard measurements of a coil, according to one embodiment.

### DETAILED DESCRIPTION

Most prior art TET circuits have utilized some form of an electromagnetic transformer and many have incorporated resonant tuning to increase the efficiency of the power transfer. The use of resonant tuned circuits in power transfer was discovered by Nikola Tesla in the early 1900's, when wireless power circuits were first demonstrated. The best analogy to resonant energy transfer in nature is the transmission of acoustic energy through space, such as a vibrating string. That is, if parallel stretched wires tuned to the same frequency are excited, the mechanical vibration is transferred from one of the wires to the adjacent wire with near perfect absorption depending on the separation distance and coupling medium. Repeater wires can be added and the acoustic power transfer range can thereby be extended. This same phenomenon occurs in magnetic resonant power transfer, where the range can be extended by precise tuning and the use of passive resonant repeaters.

The transfer of power through inductive coupled coils is not new and can be described by Maxwell's laws and especially Faraday's law. Two inductive coils can operate in near field proximity as defined by Maxwell's criterion:

$$d = \frac{λ}{2 π} \qquad ( 1 )$$

where d is distance between coils; and

λ is wavelength.

For example, at an excitation frequency of 5 MHz, the near field criterion is calculated (based on certain assumptions) at up to 9.54 meters. Typically, inductively coupled power systems will transfer power effectively from one coil to the other in the range of the diameter of the larger coil. The coupling of energy occurs by mutual inductance. That is, power from one coil is induced into the other and thereby the two coils essentially become a transformer with free space therebetween.

A transformer is an energy converter that takes an input power and converts it to an end use output power, usually with different current and voltage levels. A standard transformer converts the flow of electric current into a magnetic circuit that transfers the energy from the primary coil to the secondary coil. In transcutaneous energy transfer (TET), the primary coil is outside the body and the secondary coil is implanted inside the body. The TET transformer can be modeled as an air core transformer where the magnetic field is transferred from the primary coil to the secondary coil through free space. In the human body, the coupling actually occurs through body tissues—skin, bone, blood and interstitial fluid. The coupling circuit may include non-magnetic metal materials such as Nitinol, stainless steel and non-magnetic materials including fabric or plastic as well. As long as the coupling medium is not a ferrite or has a relative magnetic permeability less than 1, the effective primary to secondary core loss will predominately be a function of the operating frequency selected.

The various embodiments disclosed herein relate to systems having an internal resonant coil positioned inside the patient's body and an external resonant coil that is positioned outside the patient's body.

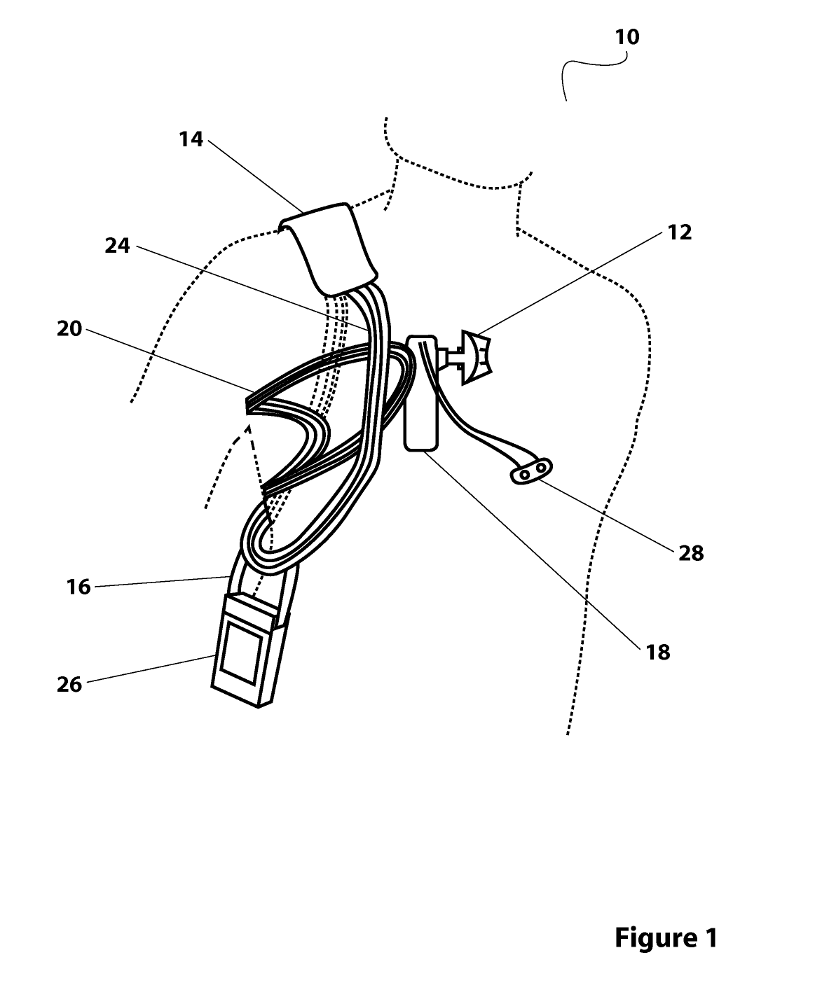

For example, FIG. 1 depicts a TET system 10, according to one embodiment, having an internal coil 20 positioned in a cavity of the patient and an external coil 24 positioned outside the patient's body. In this implementation, the internal coil 20 is positioned within the rib cage and in the pleural cavity of the patient, and specifically within the right lung cavity of the patient. Further, the external coil 24 is positioned over the shoulder and under the arm of the patient, such that the patient carries the external coil 24 much like a backpack or bag. In this embodiment, the external coil 24 has a shoulder strap cushion 14 attached to or positioned around the coil 24. The strap cushion 14 is intended to be positioned against the patient's shoulder to enhance the comfort of the patient during use of the external coil 24. The internal coil 20 is coupled to a medical device that requires power. In this exemplary embodiment, the coil 24 is coupled to an actuator 18 for a heart assist device such as an aortic cuff 12. In this embodiment, the actuator 18 is operably coupled to an ECG lead that can be used to trigger actuation of the cuff 12 by the actuator 18. Alternatively, the actuator 18 can be triggered by other methods or devices.

The external coil 24 in this embodiment is further coupled to a power source 26, which in this example is a battery 26. As shown in FIG. 1, the battery 26 is coupled to the coil 24 with two straps 16. Alternatively, the battery 26 can be coupled directly to the coil 24. The power source 26 can be a battery or household current power supply that is driven into the external coil 24. According to some embodiments, known modern TET driver circuitry is incorporated to create very efficient excitation currents. The finer the tuning of the external coil 24 to the internal coil 20, the more efficient the cross coupling of energy will be. However, if the internal coil 20 tuned frequency drifts with time due to the influences by body fluids or tissue encapsulation, the coupling efficiency will be diminished. To allow for continued power reception in the internal coil 20 with time, the internal coil 20 tuning may be sensed by a number of methods including feedback from the internal coil 20 or frequency sweeping of the external coil 24 through a narrow range of the resonant tuning. Another method to maintain effective transfer is to detune the external coil 24, thereby causing less efficient total system power use, but ensuring that the internal coil 20 will not fall out of the resonant coupling of the system. It is understood that all of the aforementioned tuning techniques are known in the design of wireless power transfer systems.

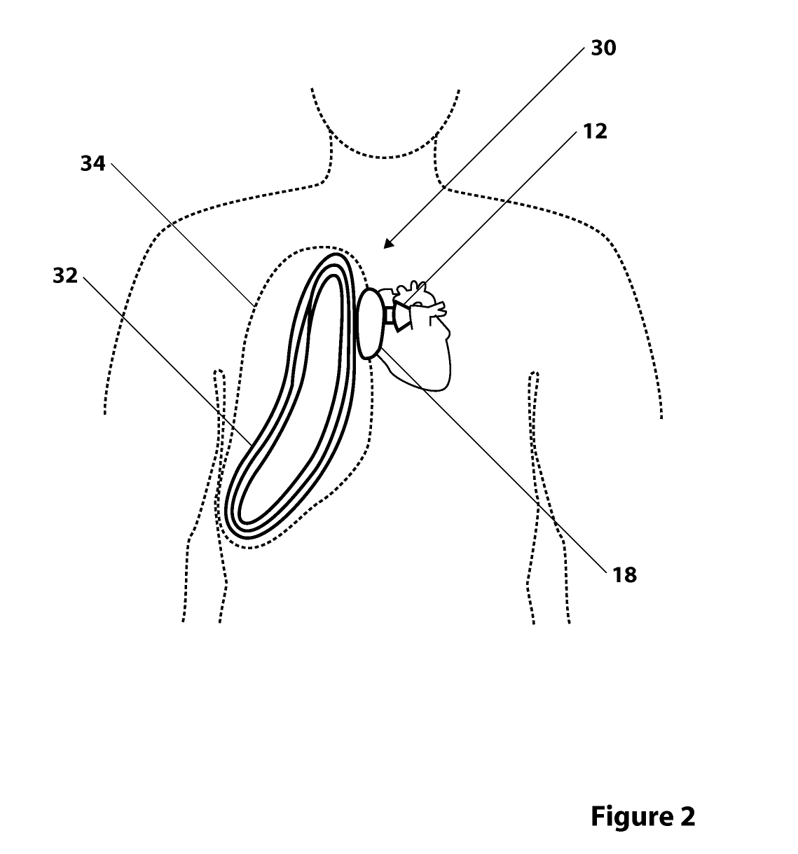

FIG. 2 depicts an alternative embodiment of a system 30 having an internal coil 32 that is positioned within the lung cavity 34 (inside the rib cage). In this particular embodiment, the coil 32 is positioned around the left lung. Alternatively, the internal coil 32 can be positioned within the pleural cavity around the right lung or around both lungs. In a further embodiment, the internal coil 32 can be positioned within the abdominal cavity or any other known cavity of the patient. In yet another alternative, the internal coil 32 can be positioned anywhere within the patient, including outside the thoracic cavity.

According to one implementation, the internal coil 32 is a self-expanding coil that, upon insertion into the target cavity, expands to its maximum diameter. In one example, the self-expanding coil is made from shape memory nitinol. Alternatively, any shape memory or self-expanding polymer or material that can be utilized in a resonant coil can be used.

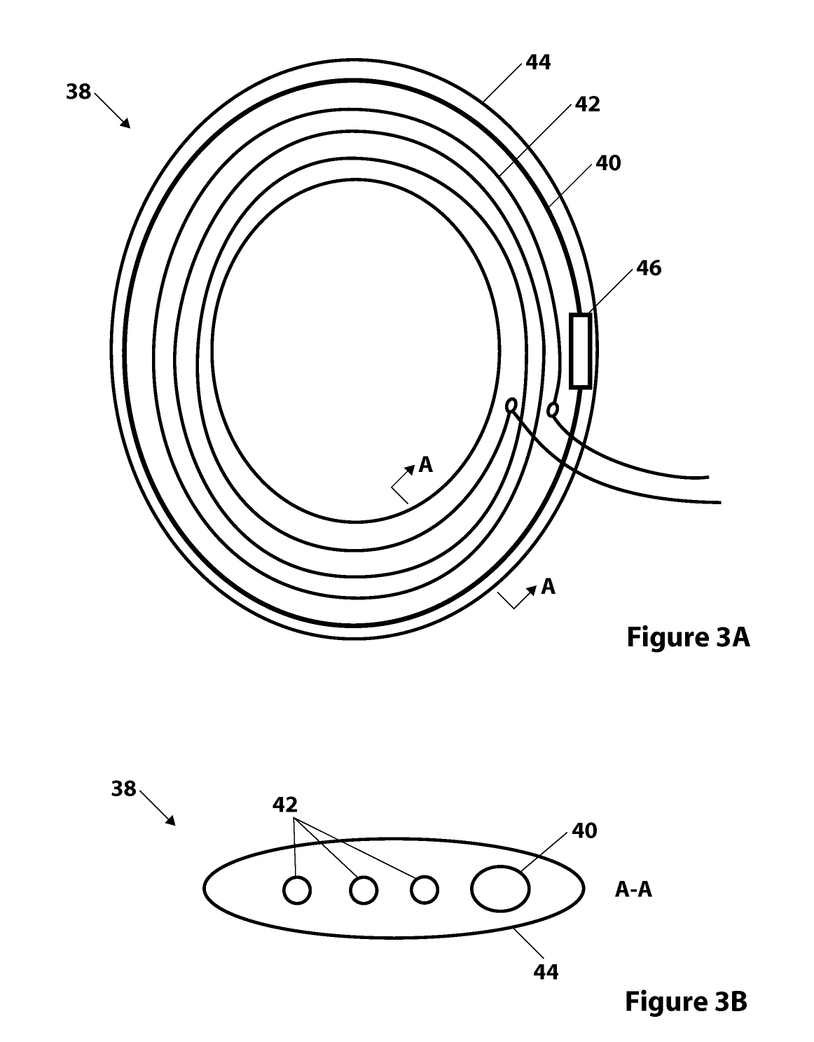

In a further alternative as shown in FIGS. 3A and 3B, an internal coil 38 can have a self-expanding structure 40 integrated into or coupled with the coil 38. FIG. 3B is a cross-section of a portion of the coil 38 at line A-A of FIG. 3A. As shown, the coil 38 has at least one coil conductor 42 and a self-expanding support structure 40 disposed within a coil casing 44. The coil conductor 42 can be a single coil component wound around the coil 38 multiple times as shown, or the conductor 42 can be two or more separate coil components. The self-expanding support structure 40 is disposed within the casing 44 and can be any shape memory material as described above. In one embodiment, the support structure 40 has an insulated connector 46 coupling two ends of the structure 40, thereby preventing the creation of an electrical circuit that competes with the circuit between the external and internal coils. As an example, a support structure that is a single loop of self-expanding nitinol cannot be used in the systems contemplated herein without an insulated connector, because the loop would create an inductive coil and thereby interfere with the electrical coupling of the external and internal coils. Alternatively, the insulated connector 46 can be any known strategic isolator or structure that creates an electrical gap (thereby preventing a conducting electrical circuit) while providing mechanical support. The insulated connector 46 can also be made of any ceramic and non-magnetic materials.

The self-expanding nature of a self-expanding coil or self-expanding coil structure can result in a self-aligning, self-securing coil that expands within the target cavity—such as the lung cavity, for example—to provide an anatomical fit which anchors itself inside the cavity. That is, in some embodiments, the coil 32 is configured to expand until it comes into contact with the inner walls of the body cavity in which it is positioned, thereby providing some frictional adherence of the coil 32 to the inner walls of the cavity or internal organs or any other structures within the cavity (thereby resulting in the “anatomical fit”). In this manner, the internal coil 32 can “match the anatomy” in the cavity. For example, in the lung cavity, the coil 32 could anchor itself against the ribs and against the lung tissue. It is understood that the relatively large space within the lung cavity can allow the coil to have a very large effective diameter, which results in greater production of energy and reduces constraints associated with anatomical mating of the internal coil 32 and the external coil (such as the external coil 24 described above or any other external coil described or contemplated herein).

In accordance with one implementation, an internal coil can have an oval shape such as the internal coil 32 shown in FIG. 2. An oval shape, according to one embodiment, is easily collapsed for easy surgical insertion into the patient such that the coil 32 can expand to its maximum diameter after insertion. Alternatively, the internal coil can have a circular shape or any other known shape for a resonant coil. Regardless of the shape, in certain embodiments the self-expanding coil 32 will generally expand to substantially match the ribs (or the interior wall of any target cavity) in shape and size.

It is understood that the geometry of the internal coil impacts the strength of the resulting magnetic field (and other energy production parameters of the coil). Hence, each of the following structural characteristics of the coil can have a resulting impact on energy production: coil diameter (or “effective diameter” for those coils that are not round), number of turns, wire diameter, and resistivity.

As discussed above, in certain embodiments, an internal coil (such as the internal coils 24, 32, or any other internal coil described or contemplated herein, for example) should be constructed to minimize the heat production of the coil and absorption of the electromagnetic energy by the patient's tissue, both of which can be harmful to the patient. In further implementations, the coil should be made of materials that are biocompatible to minimize tissue encapsulation, infection, and electrical corrosion of the electrical connections and conductors. As such, the wires can be made of silver, stainless steel, copper, gold, or any other conductive material that can be used to create an electromagnetic field receiver coil. Alternatively, conductor structures other than wires could be used in the coil, such as, for example, semiconductors or other such structures. In addition, the coil can also have a coating that is made of silicone, urethane, polyimide, Teflon or any other material that can provide electrical insulation and isolation of the receiver coil from bodily fluids. In certain alternative embodiments, the coil can be made with polyimide flex circuits or similar materials that result in high density printed circuit capabilities. In a further alternative, the coil can be constructed by weaving wires into fabrics such as Dacron or polyester.

In one implementation, the internal coil (such as the internal coils 24, 32, or any other internal coil described or contemplated herein) has a minimum diameter of at least about 6 cm. That is, even for those implementations in which the coil has an oval shape, the shortest diameter at any point on the coil is at least about 6 cm. Alternatively, the internal coil has a minimum diameter of at least about 10 cm. In a further alternative, the internal coil has a diameter ranging from about 6 cm to about 30 cm. In further implementations, the coil is an oval-shaped coil as discussed elsewhere herein with height and width as shown in FIG. 10 and discussed in further detail below. The oval-shaped coil can have a minimum height of at least about 12 cm and a minimum width of at least about 6 cm.

According to one embodiment, the wire size in the coil is selected to carry the required current and not produce significant self heating. For example, in one implementation, the wire size ranges from about 0.005 inches to about 0.75 inches. Alternatively, the wire size ranges from about AWG 0000 to about AWG 40. In one example, the wire is a Litz wire (commercially available from Cooner Wire) that is used in resonant coils construction to reduce excessive heating in alternating fields. According to one specific exemplary implementation, the wire is Cooner Litz wire PN CW4114 that is constructed of 1050 individual 44 Ga. wires arranged in 5 bundles of 5 groups of 42 wires, which creates an effective 14 AWG (1.628 mm) coil conductor. The wire is copper and is coated with polyurethane insulation bound in a Dacron outer jacket.

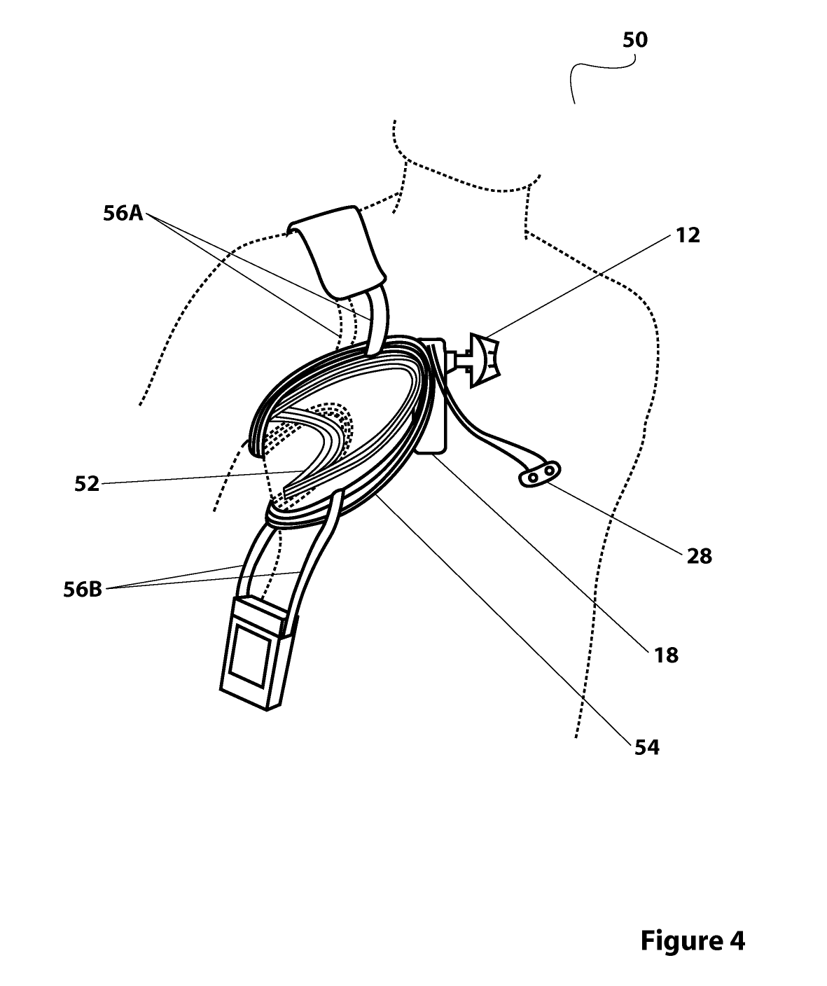

As shown in FIG. 1 and discussed above, the outer coil 24 can be configured as a shoulder strap having a shoulder strap cushion 14. That is, the coil 24 has a padded or cushioned shoulder strap cushion 14 that can be positioned on the patient's shoulder like a strap on a bag or purse. Alternatively, the outer coil can be coupled or physically joined to another supporting structure as a combination feature, such as the exemplary embodiment of a system 50 depicted in FIG. 4 in which the external coil 54 is coupled to one or more straps 56A, 56B as shown. That is, the shoulder straps 56A are coupled to an upper portion of the coil 54, while the lower straps 56B are coupled to a lower portion of the coil 54.

In a further alternative, the outer coil can be configured in any number of ways, such as, for example, sewn into a vest, shirt, jacket, or other article of clothing, attached to a belt, or incorporated into a bed, chair, or car seat. In yet another alternative, the outer coil can have any physical coil configuration that creates a desired effective area and electrical impedance. In a further implementation, the outer coil can be any coil that is not required to be adhered to the patient's skin. The outer coil can be made of any of the same materials as described with respect to the inner coil embodiments disclosed herein.

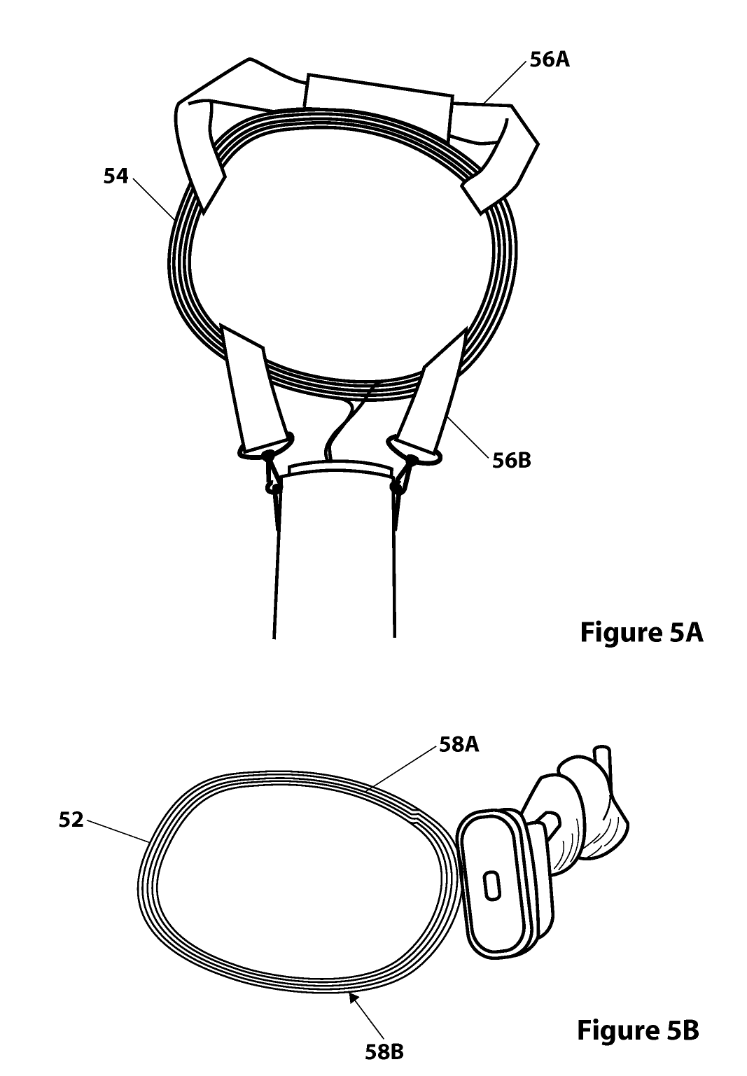

FIGS. 4, 5A, and 5B depict another embodiment of a TET system 50. This system 50 has an external coil 54 as best shown in FIG. 5A and an internal coil 52 as best shown in FIG. 5B. In use as best shown in FIG. 4, the external coil 54 is maintained in close proximity to the internal coil 52. For example, the external coil 54 can be configured to be positioned outside of the patient's body in a configuration that puts the entire coil 54 as close to the entire internal coil 52 as possible. The closer the two coils 52, 54 are positioned to each other, the more efficient the energy transfer is between them, and thus the less system power is required to generate the amount of energy necessary to power the desired medical device.

According to one embodiment, any internal coil embodiment described or contemplated herein—including the coil 52 depicted in FIG. 5B—can have two sides, each having a coating made of different materials. For example, the internal coil 52 has a first side (or “external side”) 58A of the coil can have a coating that is adherent, while the second side (or “internal side”) 58B of the coil has a coating that is lubricious or slippery. In one embodiment, the lubricious coating on the internal side 58B is intended to contact the lungs, while the adherent, flexible materials on the external side 58A are intended to contact the rib cage, thereby resulting in a coil 52 that is stable and comfortable. More specifically, the slipperiness of the lubricious coating on the internal side 58B is intended to not cause damage to the internal organs (such as the lungs) when the coating comes into contact with such organs, while the stickiness of the adherent coating on the external side 58A is intended to enhance the adherence of the coil 52 to the rib cage or other internal wall of the target cavity, thereby providing some stability or fixation of the coil 52.

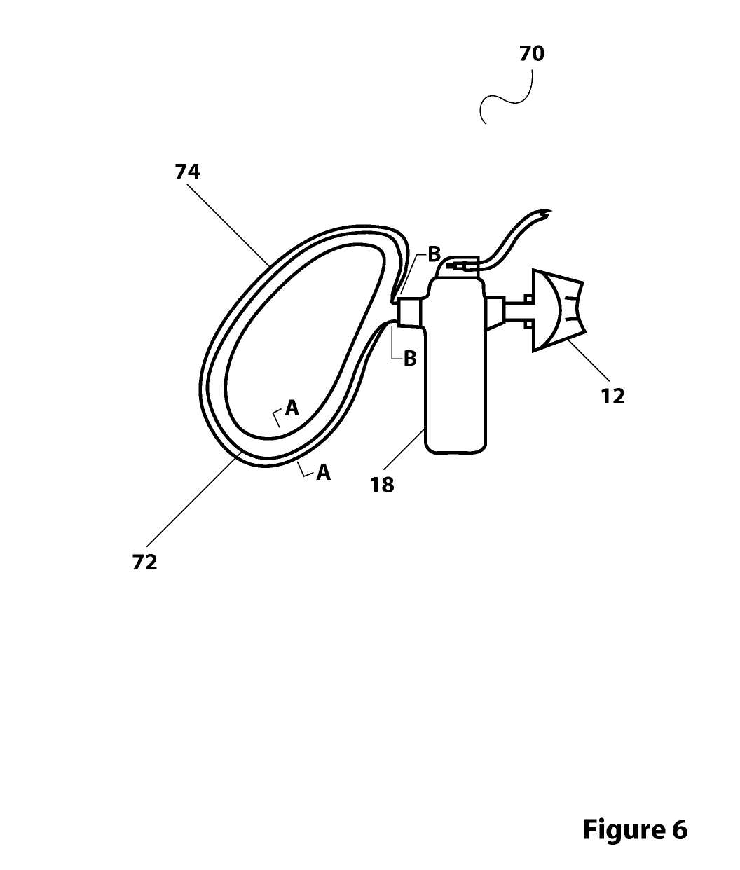

FIG. 6 depicts an implantable TET-powered heart assist system 70, according to one implementation. This system is configured to provide heart support by counterpulsation and pumping of the ascending aorta in a manner as described in U.S. Pat. No. 6,808,484. The system has a pulsatile pump 18 that is powered by an internal resonant coil 72, wherein both the pump 18 and the coil 72 are located in the lung or thoracic cavity. In addition to generating power, the internal coil 72 in this example also has a compliance chamber 74 disposed around or adjacent to the wires of the coil 72. In the embodiment as shown in FIG. 6, the compliance chamber 74 is positioned along and coupled to the outer edge of the coil 72. The compliance chamber 74 is configured to store the pumping fluid during the deflation stroke of the pump 18. A typical inflation fluid for an implantable pump such as pump 18 would be a silicone fluid such as polydimethysiloxane with a viscosity in the range of 1 to 40 cSt. The fluid that is moved out of the pump 18 into the cuff 12 can also be moved into the compliance chamber (also called a “storage chamber”) 74.

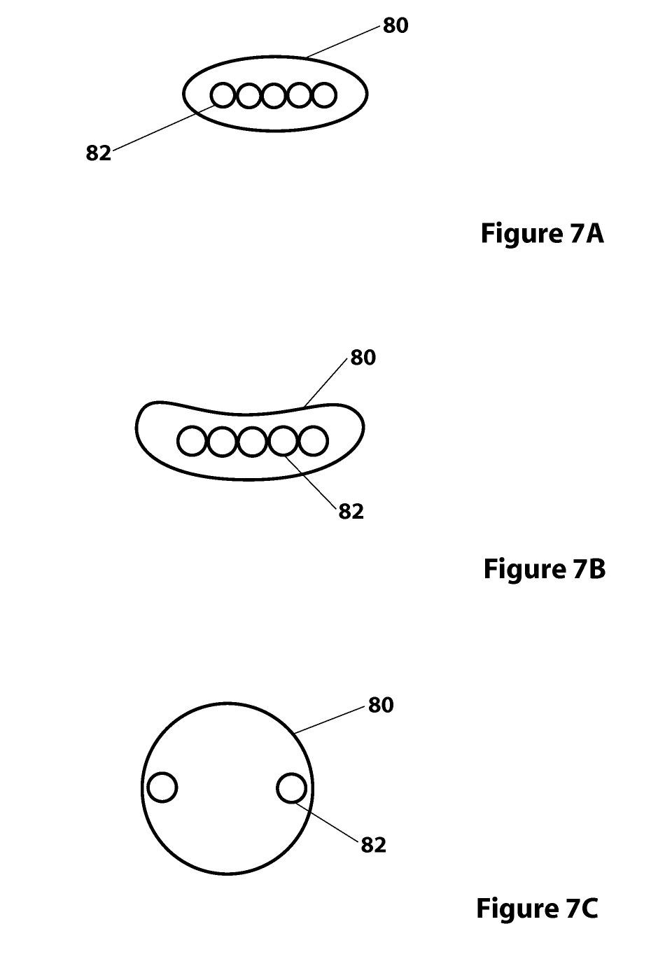

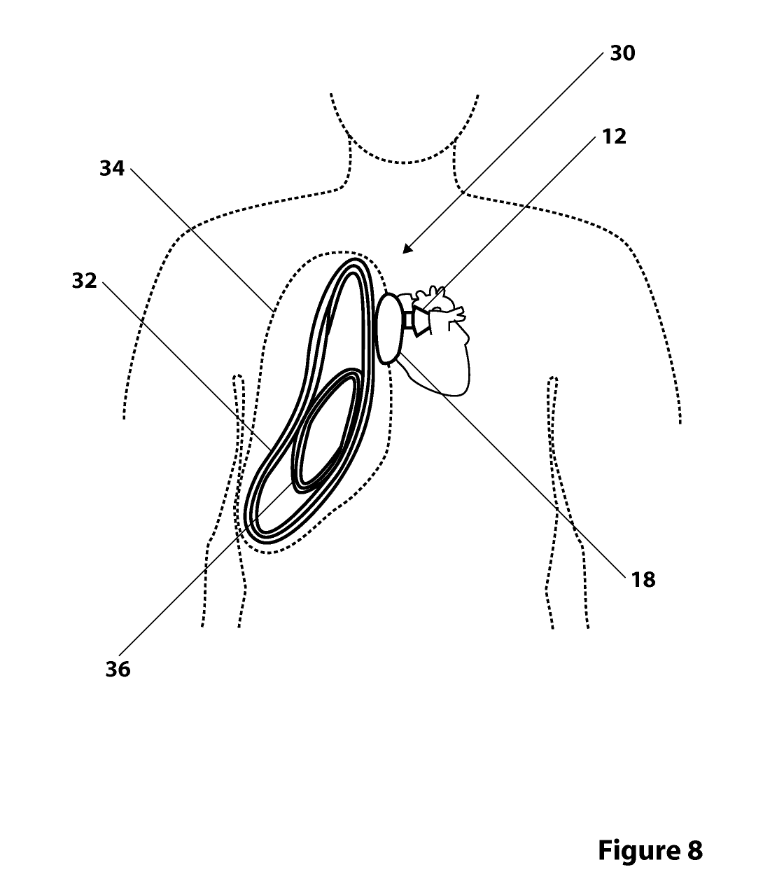

Alternatively, the compliance chamber 74 need not be coupled to an outer edge of the coil 72. Instead, as shown in FIGS. 7A and 7B (which depicts a cross-section of an alternative embodiment along the AA cross-section line at a location as shown in the similar coil 72 of FIG. 6) and FIG. 7C (which depicts a cross-section of an alternative embodiment along the BB cross-section line at a location as shown in the similar coil 72 of FIG. 6), the compliance chamber 80 is disposed around the coil 82. As such, when the inflation fluid is moved into the compliance chamber 80, the compliance chamber 80 expands into an expanded state (or “expansion state”) as shown in FIG. 7A. And when the inflation fluid is moved out of the chamber 80, the chamber 80 deflates into a deflated state (or “deflation state”) as shown in FIG. 7B. In certain embodiments, the compliance chamber 80 can also function as a heat transfer mechanism that helps to cool the coil 82, which can be particularly helpful for systems requiring high power such as an artificial heart. In a further alternative embodiment, the internal coil can incorporate fins or folds to increase the surface area for cooling.

The various embodiments disclosed herein generate energy that is sufficient to power any implantable medical device. That is, some combination of the large external and internal coils, the positioning of those coils with respect to each other (including the positioning of the internal coil in a patient cavity such as the lung cavity), and various other characteristics of the systems can result in energy generation that is more than sufficient for any known implantable medical device or device that is positioned on the patient's body. For example, the systems contemplated herein can power any drug delivery device, any CRM device, any heart assist device, or any other known implantable device. Alternatively, the various systems disclosed herein can also be used to power smaller devices, including, for example, devices intended for use in the eyes or ears of a patient.

In another implementation, resonant power can also incorporate passive resonant coils (also referred to as “repeater coils”) to increase effective coupling distance between the transmitter and ultimate receiver coil. For example, an external coil could be attached to the patient in the form of a vest, shoulder strap, or anatomical adhesive attachment, wherein the coil is not connected to a battery transmitter circuit. This offers a separation distance advantage from the heavy power generating transmitter coil without significant loss, since the repeater coils are tuned to the exact resonant frequency of the transmitter and receiver coils. Since the excitation energy is transferred to the passive coil causing it to create and collapse the energy field very efficiently, the repeater coils can be used both outside and inside the body.

One exemplary embodiment of a system having a repeater coil 36 is depicted in FIG. 8. This system is similar to the system depicted in FIG. 2, with an internal coil 32. In addition, this system also includes a repeater coil 36. In the implementation shown, the repeater coil 36 is adhered to the chest of the patient in close proximity to the internal coil 32 disposed within the patient's lung cavity 34.

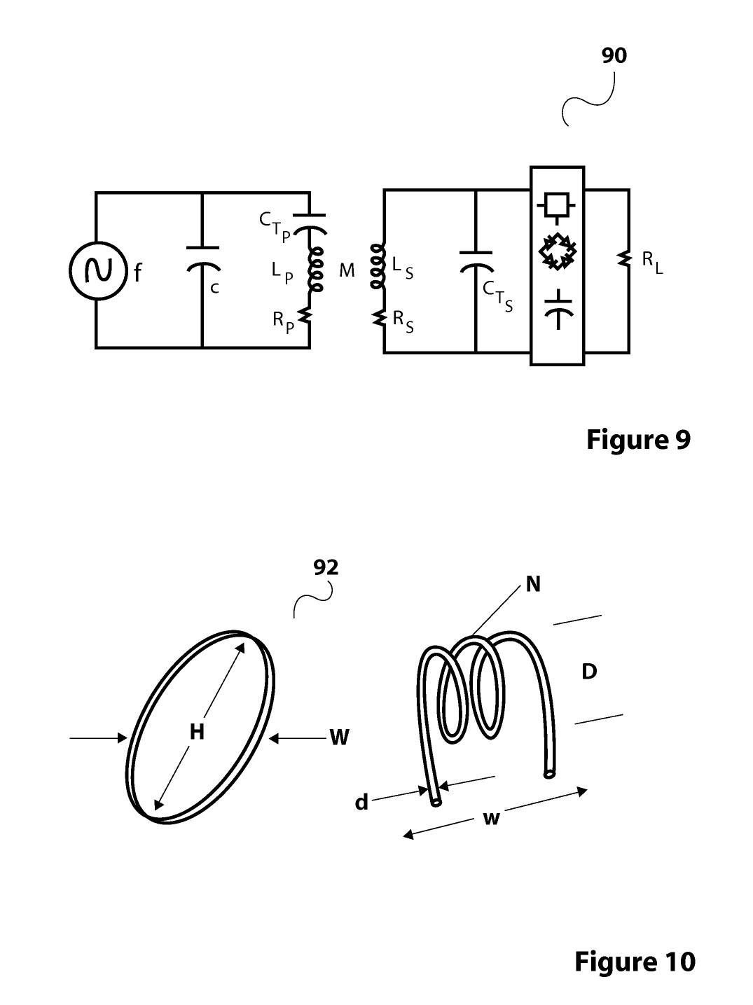

A passive repeater coil is constructed via the same techniques as the receiver coils, but is self-contained electrically in that it is a closed loop circuit. A circuit description can be seen in FIG. 9 as the secondary capacitor C_{Ts} and inductor Lₛ, without the the rectifier control circuit 90 and the load R_{L}. In one alternative implementation, a series of passive repeater coils can be envisioned that would function as a distribution network between final use destinations. For example, a repeater coil in the lungs could excite several pacing coils located in the heart as described in U.S. Published Application 2009/0204170 (Hastings), which is hereby incorporated herein by reference in its entirety. As previously described in Hastings, multiple receiver coils may be excited simultaneously, since the resonant energy will be transferred to any coil tuned to the same resonant frequency.

While multiple embodiments are disclosed, still other embodiments of the present invention will become apparent to those skilled in the art from the following detailed description, which shows and describes illustrative embodiments of the invention. As will be realized, the invention is capable of modifications in various obvious aspects, all without departing from the spirit and scope of the present invention. Accordingly, the drawings and detailed description are to be regarded as illustrative in nature and not restrictive. Further, although the present invention has been described with reference to preferred embodiments, persons skilled in the art will recognize that changes may be made in form and detail without departing from the spirit and scope of the invention.

### EXAMPLE

In one example, the system has an internal coil similar to the coil 20 depicted in FIG. 2. In this example, the coil 20 is 15 cm in width (W) and 25 cm in height (H) as shown in a basic coil 92 description in FIG. 10. The electrical model of the TET system in this example is shown in FIG. 9 and is a classic description of an air core transformer with a signal source and a resonant tuned transmitter coupled by a coupling coefficient M to secondary internal receiver with a resonant tuning and rectifier and control circuitry 90. The operating frequency is largely determined by analysis of tissue absorption and allowed exposure safety limits from regulatory agencies and is discussed further in the exemplary coil design for the test system that follows.

The effective diameter for this exemplary coil can be determined from the effective coil area. Based on certain simple assumptions, the effective coil diameter of the exemplary coil may be determined by calculating the area of the resulting anatomical coil geometry and resolving that into an equivalent circular radius. In the case of the internal coil having a width of 15 cm and a height of 25 cm, the effective coil area of 295 cm² (π×r1×r2) can be equated to a circular coil (A=π×r²) where the effective radius would be 9.7 cm.

The inductance and resistance of the coil is:

L=2 μₒDN² and R=ρNπD/α;

Where μₒ=permittivity for free space, 4 π×10⁻⁷

D=effective diameter;

N=numbers of turns;

ρ=resistivity Ω−m;

α=wire cross sectional area m²;

and Q=4(/ρ)fN α; and

Where f=tuned frequency.

The selected operating frequency in human tissue must be above 125 kHz to prevent DC tissue stimulation and below 10 Mhz to minimize direct EM tissue heating described by the Specific Adsorption Rate (SAR) limits established by the ICNIRP. The current design example will be based on a 1 MHz design frequency.

The power requirements for heart assist devices is in the range 1 to 10 watt average. Assuming the voltage required to drive a motor or actuator is in the range 10 to 15 volts, a design voltage of 12 v will be used in this example. To keep heating below 40 mW/cm², a Litz wire diameter is selected of a large enough diameter and high enough wire count to minimize Eddy currents and DC resistive heating. For this example 2 mm diameter Litz wire constructed with 5 bundles of 5/42/44 wire strands will utilized. The effective diameter will be 2 mm and the resistivity will be copper in this example but may also be silver or gold if desired for medical implant requirements.

The inner receiver coil 20 will be constructed from 6 turns of 2 mm wire coiled to give an effective diameter of r=97 mm. Solving for L and C is as follows:

F=ω/2π=1/2 π√LC

L_{eff}=12.018 μH

X_{eff}=75.5Ω

R_{eff}=0.159Ω

C=(1/2 πf)²/L

C=2107.7 pf

Q=1/R√LC

Q_{eff}=473.3

The outer transmit receiver coil 24 will be constructed from 10 turns of 2 mm wire coiled to give an effective diameter of r=158 mm. Solving for L and C is as follows:

F=ω/2π=1/2 π√LC

L_{eff}=49.009 μH

X_{eff}=307.9Ω

R_{eff}=0.557Ω

C=(1/2 πf)²/L

C=516.8 pf

Q=1/R√LC

Q_{eff}=552.3

The TET system in this example is configured to deliver power for a 3 W-6 W device powered at 12 volts, meaning that this system can power such devices as the heart assist device described in U.S. Pat. No. 6,808,484, which is hereby incorporated herein by reference in its entirety. It is understood that similar configurations can be created which deliver less power and can occur at greater separation distances. It is also understood that multiple receiver coils of identical design can be created to power multiple devices. One possible advantage of a cavity coil design or supported loop design is that looser coupling can occur between the primary and secondary coils. Patient comfort and safety and quality of life can be improved with the designs of this invention.

What is claimed is:

1. A transcutaneous energy transfer system comprising:
(a) an internal coil sized to be positioned within a pleural cavity of a patient, wherein the internal coil is configured to be positioned in proximity with at least one lung within the pleural cavity; and
(b) an external coil configured to be positioned in proximity to the patient such that the external coil and the internal coil are inductively coupled.

2. The system of claim 1, wherein the internal coil has a diameter of at least about 6 cm.

3. The system of claim 1, wherein the internal coil is positioned substantially against an inner wall of the pleural cavity.

4. The system of claim 1, further comprising a self-expanding structure operably coupled to the internal coil.

5. The system of claim 4, wherein the self-expanding structure is made of a shape memory material.

6. The system of claim 4, wherein the self-expanding structure is configured to expand such that the internal coil is in contact with an inner wall of the pleural cavity.

7. The system of claim 4, wherein the self-expanding structure comprises at least one insulated connector configured to prevent formation of a competing electrical circuit.

8. The system of claim 1, wherein the internal coil is configured to be positioned around the at least one lung.

9. The system of claim 1, wherein the external coil comprises a shoulder strap cushion.

10. The system of claim 1, wherein the external coil is integrated into a shoulder strap, backpack, bag, vest, shirt, jacket, bed, chair, or car seat.

11. A transcutaneous energy transfer system operably coupled to a heart assist system, the transcutaneous energy transfer system comprising:
(a) an internal coil sized to be positioned within a pleural cavity of a patient;
(b) a compliance chamber associated with the internal coil; and
(c) an external coil configured to be positioned in proximity to the patient such that the external coil and the internal coil are inductively coupled.

12. The system of claim 11, wherein the compliance chamber is coupled to the internal coil.

13. The system of claim 11, wherein the internal coil is positioned within the compliance chamber.

14. The system of claim 11, wherein the compliance chamber is an expandable compliance chamber having an inflated configuration and a deflated configuration.

15. A transcutaneous energy transfer system comprising:
(a) an internal coil sized to be positioned within a pleural cavity of a patient;
(b) a repeater coil configured to be positioned in proximity to the patient such that the repeater coil and the internal coil are inductively coupled; and
(c) an external coil configured to be positioned such that the external coil and the repeater coil are inductively coupled, whereby the external coil is inductively coupled to the internal coil.

16. The system of claim 15, wherein the repeater coil is positioned in proximity to skin of the patient.

17. The system of claim 15, wherein the internal coil has a diameter of at least about 6 cm.

18. The system of claim 15, further comprising a self-expanding structure operably coupled to the internal coil.

19. The system of claim 15, wherein the external coil comprises a shoulder strap cushion.

20. The system of claim 15, wherein the external coil is integrated into a shoulder strap, backpack, bag, vest, shirt, jacket, bed, chair, or car seat.
